(19) United States
(12) Patent Application Publication
(10) Pub. No.: US 2011/0150767 A1
(43) Pub. Date: Jun. 23, 2011

(54) ALPHA-SUBSTITUTED AND ALPHA-UNSUBSTITUTED AROMATIC AMINO ACID DERIVATIVES AND COMPOSITIONS THEREOF FOR USE TO TREAT, DIAGNOSE, OR MONITOR A MEDICAL CONDITION

(76) Inventors: Michael F. Wempe, Aurora, CO (US); Hitoshi Endou, Tokyo (JP)

(21) Appl. No.: 12/969,690

(22) Filed: Dec. 16, 2010

Related U.S. Application Data
(60) Provisional application No. 61/287,505, filed on Dec. 17, 2009.

(51) Int. Cl. A61K 49/00; C07D 213/02; A61K 31/435; A61P 35/00; A61J 1/00

(52) U.S. Cl. 424/9.1; 546/339; 514/277; 604/403; 206/459.5

(57) ABSTRACT

A compound represented by the general formula 1 as described, a pharmaceutically acceptable salt or ester thereof, a solvate thereof, a chelate thereof, a non-covalent complex thereof, a pro-drug thereof, a radio-labeled analog thereof (i.e. ¹⁸F, ¹²⁴I, ¹¹C, etc), and mixtures of any of the foregoing, for diagnosis or monitoring of a medical condition.

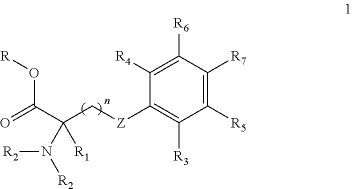

### CROSS-REFERENCE TO RELATED APPLICATIONS

This claims the benefits of U.S. Provisional Application No. 61/287,505, filed on Dec. 17, 2009, the disclosure of which is incorporated by reference in its entirety.

### BACKGROUND OF INVENTION

### Field of the Invention

Cancer cells have an abnormally rapid uptake of amino acids; consequently, they tend to proliferate much faster than normal cells. L-type amino acid transporter 1 (LAT-1) has been shown to be a cancer-specific membrane protein (Kanai Y et al., Journal of Biological Chemistry, 1998, 273, 23629-23632); whereas, L-type amino acid transporter 2 (LAT-2) exists in normal cells. It has been shown that LAT-1 specifically transports neutral branched-chain amino acids and aromatic acids (Uchino et al., Molecular Pharmacology, 2002, 61, 729-737); whereas, LAT-2 transports essential amino acids. Therefore, as described by Endou et al. in the 2008 U.S. Pat. No. 7,345,068 B2, compounds that selectively inhibit L-type amino acid transporter 1 offer researchers with a novel cancer molecular target. In addition for use as a therapy to treat disease, compounds selective for LAT1 and/or LAT2 afford novel materials for use not only to diagnosis and/or monitor diseases. (i.e. cancer), but also to evaluate suitable doses of LAT1-inhibitable drugs in individual patients as selective imaging probes (i.e. positron emission tomography/computed tomography (PET/CT) probes). It is the object of this invention to provide compounds and compositions.

### BRIEF SUMMARY OF THE INVENTION

A first embodiment according to the present invention concerns a composition, comprising at least one compound represented by formula 1:

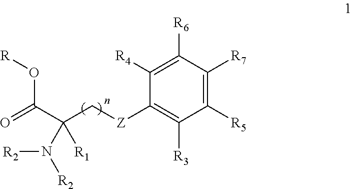

a pharmaceutically acceptable salt or ester thereof, a solvate thereof, a chelate thereof, a non-covalent complex thereof, a pro-drug thereof, a radio-labeled analog thereof (i.e. ¹⁸F, ¹²⁴I, ¹¹C, etc), and mixtures of any of the foregoing, wherein:
R is selected from hydrogen, C₁-C₂₂ alkyl, C₄-C₂₂ alkenyl, C₄-C₂₀ dienyl, C₆-C₂₂ trienyl, C₈-C₂₂ tetraenyl, a polyethylene glycol, a polypropylene glycol, or co-blocked polymer;
R¹ is selected from hydrogen, deuterium, tritium, methyl (—CH₃), —CH₂F, —CHF₂, or —CF₃;
R² is individually selected from hydrogen, methyl, or ethyl;
n is 0, 1, 2, or 3 methylene (—CH₂—) units;
Z is selected from O, N, NR′, S, SO, SO₂, —SO₂NR′—, —NR′SO₂—, wherein R′ is selected from hydrogen, alkyl, substituted alkyl, cycloalkyl, substituted cycloalkyl, aryl, substituted aryl, aralkyl and substituted aralkyl; or even where Z is absent (when n=0 and Z absent, then directly connected to the aromatic ring).
R³ and R⁴ are individually selected from hydrogen, deuterium, tritium, —Cl, —Br, —I, —F, —OH, —OR′, C1-C4 alkyl or substituted alkyl, —NH₂, —NHR′, —N(R′)₂, —NHSO₂R′, —NR₈SO₂R′, —SO₂NH₂, —SO₂NHR′, —SO₂N(R′)₂, wherein R′ has been previously defined;
R⁵, R⁶, and R⁷ are individually selected from hydrogen, deuterium, tritium, —Br, —I, —F, —OH, —OR′, alkyl and substituted alkyl (C1-C4), —NH₂, —NHR′, —N(R′)₂, —NHSO₂R′, —NR′SO₂R′, —SO₂NH₂, —SO₂NHR′, —SO₂N(R′)₂, wherein R′ has been previously defined; or from the group consisting of C₃-C₂₄ alkyl, C₃-C₂₄ alkenyl, C₄-C₂₄ dienyl, C₆-C₂₄ trienyl, C₈-C₂₄ tetraenyl and mixtures thereof, C₆-C₁₈ aryl, substituted C₆-C₁₈ aryl, C₁-C₁₄-alkoxy, halogen, carboxy, cyano, C₁-C₁₄-alkanoyloxy, C₁-C₁₄-alkylthio, C₁-C₁₄-alkylsulfonyl, C₂-C₁₄-alkoxycarbonyl, C₂-C₁₄-alkanoylamino, —O—R⁸, S—R⁸, —SO₂—R⁸, —NHSO₂R⁸ and —NHCO₂R⁸, wherein R⁸ is phenyl, naphthyl, or phenyl or naphthly substituted with one to three groups selected from C₁-C₆-alkyl, C₆-C₁₀ aryl, C₁-C₆-alkoxy and halogen, and C₄-C₂₀ hydroxyheteroaryl wherein the heteroatoms are selected from the group consisting of sulfur, nitrogen, and oxygen;

Another embodiment concerns a method to diagnosis and/or monitor a condition comprising administering an effective amount of a composition comprising a compound, pharmaceutical and/or dermatological carriers, wherein the compound is represented by the general formula 1:

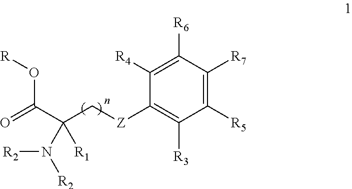

a pharmaceutically acceptable salt or ester thereof, a solvate thereof; a chelate thereof, a non-covalent complex thereof, a pro-drug thereof, a radio-labeled analog thereof (i.e. ¹⁸F, ¹²⁴I, ¹¹C, etc), and mixtures of any of the foregoing, wherein:
R is selected from hydrogen, C₁-C₂₂ alkyl, C₄-C₂₂ alkenyl, C₄-C₂₀ dienyl, C₆-C₂₂ trienyl, C₈-C₂₂ tetraenyl, a polyethylene glycol, a polypropylene glycol, or co-blocked polymer;
R¹ is selected from hydrogen, deuterium, tritium, methyl (—CH₃), —CH₂F, —CHF₂, or —CF₃;
R² is individually selected from hydrogen, methyl, or ethyl;
n is 0, 1, 2, or 3 methylene (—CH₂—) units;
Z is selected from O, N, NR′, S, SO, SO₂, —NR′SO₂—, wherein R′ is selected from hydrogen, alkyl, substituted alkyl, cycloalkyl, substituted cycloalkyl, aryl, substituted aryl, aralkyl and substituted aralkyl; or even where Z is absent (when n=0 and Z absent, then directly connected to the aromatic ring).
R³ and R⁴ are individually selected from hydrogen, deuterium, tritium, —Cl, —Br, —I, —F, —OH, —OR′, C₁-C₄ alkyl or substituted alkyl, —NH₂, —NHR′, —N(R′)₂, —NHSO₂R′, —NR₈SO₂R′, —SO₂NH₂, —SO₂NHR′, —SO₂N(R′)₂, wherein R′ has been previously defined;
R⁵, R⁶, and R⁷ are individually selected from hydrogen, deuterium, tritium, —Cl, —Br, —I, —F, —OH, —OR′, alkyl and substituted alkyl (C1-C4), —NH₂, —NHR′, —N(R′)₂, —NHSO₂R′, —NR′SO₂R′, —SO₂NH₂, —SO₂NHR′, —SO₂N(R′)₂, wherein R′ has been previously defined, or from the group consisting of C₃-C₂₄ alkyl, C₃-C₂₄ alkenyl, C₄-C₂₄ dienyl, C₆-C₂₄ trienyl, C₈-C₂₄ tetraenyl and mixtures thereof, C₆-C₁₈ aryl, substituted C₆-C₁₈ aryl, C₁-C₁₄-alkoxy, halogen, carboxy, cyano, C₁-C₁₄-alkanoyloxy, C₁-C₁₄-alkylthio, C₁-C₁₄-alkylsulfonyl, C₂-C₁₄-alkoxycarbonyl, C₂-C₁₄-alkanoylamino, —O—R⁸, S—R⁸, —SO₂—R⁸, —NHSO₂R⁸ and —NHCO₂R⁸, wherein R⁸ is phenyl, naphthyl, or phenyl or naphthly substituted with one to three groups selected from C₁-C₆-alkyl, C₆-C₁₀ aryl, C₁-C₆-alkoxy and halogen, and C₄-C₂₀ hydroxyheteroaryl wherein the heteroatoms are selected from the group consisting of sulfur, nitrogen, and oxygen;

### DETAILED DESCRIPTION

The present invention concerns a series of novel compounds and their compositions which are represented by the general formula 1:

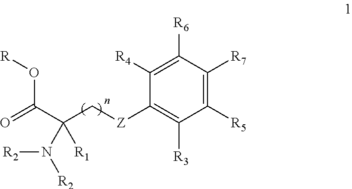

a pharmaceutically acceptable salt or ester thereof, a solvate thereof, a chelate thereof, a non-covalent complex thereof, a pro-drug thereof, a radio-labeled analog thereof (i.e. ¹⁸F, ¹²⁴I, ¹¹C, etc), and mixtures of any of the foregoing, wherein:
R is selected from hydrogen, C₁-C₂₂ alkyl, C₄-C₂₂ alkenyl, C₄-C₂₀ dienyl, C₆-C₂₂ trienyl, C₈-C₂₂ tetraenyl, a polyethylene glycol, a polypropylene glycol, or co-blocked polymer;
R¹ is selected from hydrogen, deuterium, tritium, methyl (—CH₃), —CH₂F, —CHF₂, or —CF₃;
R² is individually selected from hydrogen, methyl, or ethyl;
n is 0, 1, 2, or 3 methylene (—CH₂—) units;
Z is selected from O, N, NR′, S, SO, SO₂, —SO₂NR′—, —NR′SO₂—, wherein R′ is selected from hydrogen, alkyl, substituted alkyl, cycloalkyl, substituted cycloalkyl, aryl, substituted aryl, aralkyl and substituted aralkyl; or even where Z is absent (when n=0 and Z absent, then directly connected to the aromatic ring).
R³ and R⁴ are individually selected from hydrogen, deuterium, tritium, —Cl, —Br, —I, —F, —OH, —OR′, C1-C4 alkyl or substituted alkyl, —NH₂, —NHR′, —N(R′)₂, —NHSO₂R′, —NR₈SO₂R′, —SO₂NH₂, —SO₂NHR′, —SO₂N(R′)₂, wherein R′ has been previously defined;
R⁵, R⁶, and R⁷ are individually selected from hydrogen, deuterium, tritium, —Cl, —Br, —I, —F, —OH, —OR′, alkyl and substituted alkyl (C1-C4), —NH₂, —NHR′, —N(R′)₂, —NHSO₂R′, —NR′SO₂R′, —SO₂NH₂, —SO₂NHR′, —SO₂N(R′)₂, wherein R′ has been previously defined; or from the group consisting of C₃-C₂₄ alkyl, C₃-C₂₄ alkenyl, C₄-C₂₄ dienyl, C₆-C₂₄ trienyl, C₈-C₂₄ tetraenyl and mixtures thereof, C₆-C₁₈ aryl, substituted C₆-C₁₈ aryl, C₁-C₁₄-alkoxy, halogen, carboxy, cyano, C₁-C₁₄-alkanoyloxy, C₁-C₁₄-alkylthio, C₁-C₁₄-alkylsulfonyl, C₂-C₁₄-alkoxycarbonyl, C₂-C₁₄-alkanoylamino, —O—R⁸, S—R⁸, —SO₂—R⁸, —NHSO₂R⁸ and —NHCO₂R⁸, wherein R⁸ is phenyl, naphthyl, or phenyl or naphthly substituted with one to three groups selected from C₁-C₆-alkyl, C₆-C₁₀ aryl, C₁-C₆-alkoxy and halogen, and C₄-C₂₀ hydroxyheteroaryl wherein the heteroatoms are selected from the group consisting of sulfur, nitrogen, and oxygen;

As used throughout this application, the term “pharmaceutically effective amount of a compound for pharmaceutical use” shall mean the amount of administered compound required to exhibit the diagnostic effect. Examples of methods of administration include, but are not limited to, oral administration (e.g., ingestion, buccal or sublingual administration), anal or rectal administration, topical application, aerosol application, inhalation, intraperitoneal administration, intravenous administration, transdermal administration, intradermal administration, subdermal administration, intramuscular administration, intrauterine administration, vaginal administration, administration into a body cavity, surgical administration (for example, at the location of a tumor or internal injury), administration into the lumen or parenchyma of an organ, and parenteral administration. The compositions can be administered in any form by any means. Examples of forms of administration include, but are not limited to, injections, solutions, creams, gels, implants, ointments, emulsions, suspensions, microspheres, powders, particles, microparticles, nanoparticles, liposomes, pastes, patches, capsules, suppositories, tablets, transdermal delivery devices, sprays, suppositories, aerosols, or other means familiar to one of ordinary skill in the art. In some embodiments, the compositions can be combined with other components. Examples include, but are not limited to, coatings, depots, matrices for time release and osmotic pump components.

The term “solvate” refers to the compound formed by the interaction of a solvent and a compound. Suitable solvates are pharmaceutically acceptable solvates, such as hydrates, including monohydrates and hemi-hydrates. “Pharmaceutically acceptable salt” refers to a salt of a compound that is pharmaceutically acceptable and that possesses the desired pharmacological activity of the parent compound. Such salts may include: (i) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, and the like; or (ii) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

In some embodiments, the one or more compounds, or compositions of the present invention, are administered to persons or animals to provide substances in any dose range that will produce desired diagnostic results. Dosage will depend upon the substance or substances administered, the diagnostic endpoint desired, the desired effective concentration at the site of action or in a body fluid, and the type of administration. Information regarding appropriate doses of substances are known to persons of ordinary skill in the art and may be found in references such as L. S. Goodman and A. Gilman, eds, The Pharmacological Basis of Therapeutics, Macmillan Publishing, New York, and Katzung, Basic & Clinical Pharmacology, Appleton & Lang, Norwalk, Conn. (6.sup.th Ed. 1995). In some embodiments, the compounds and compositions of the present invention may be administered to a subject. Suitable subjects include a cell, population of cells, tissue or organism. In certain embodiments, the subject is a mammal such as a human. The compounds may be administered in vitro or in vivo.

The invention includes methods in which one or more compounds are an admixture or otherwise combined with one or more compounds and may be in the presence or absence of commonly used excipients; for example, but not limited to: i) diluents and carriers such as starch, mannitol, lactose, dextrose, sucrose, sorbitol, mannitol, cellulose, and the like; ii) binders such as starch paste, gelatin, magnesium aluminum silicate, methylcellulose, alginates, gelatin, sodium carboxymethyl-cellulose, polyvinylpyrrolidone and the like; iii) lubricants such as stearic acid, talcum, silica, polyethylene glycol, polypropylene glycol and the like; iv) absorbents, colorants, sweeteners and the like; v) disintegrates, (e.g., calcium carbonate and sodium bicarbonate) such as effervescent mixtures and the like; vi) excipients (e.g. cyclodextrins and the like); vii) surface active agents (e.g., cetyl alcohol, glycerol monostearate), adsorptive carriers (e.g., kaolin and bentonite), emulsifiers and the like. Examples of carriers include, without limitation, any liquids, liquid crystals, solids or semi-solids, such as water or saline, gels, creams, salves, solvents, diluents, fluid ointment bases, ointments, pastes, implants, liposomes, micelles, giant micelles, and the like, which are suitable for use in the compositions.

Furthermore, said invention includes compositions prepared using conventional mixing, granulating, or coating methods and may contain 0.0001 to 90% of the active ingredients. In some embodiments, the one or more compounds are for pharmaceutical use or for diagnostic use. Such methods can be used, for example, to prepare a bio-enhanced pharmaceutical composition in which the solubility of the compound(s) is (are) enhanced. In some embodiments, the resulting compositions contain a pharmaceutically effective amount of a compound for diagnostic use. The resulting compositions (formulations) may be presented in unit dosage form and may be prepared by methods known in the art of pharmacy. All methodology includes the act of bringing the active ingredient(s) into association with the carrier which constitutes one or more ingredients. Therefore, compositions (formulations) are prepared by blending active ingredient(s) with a liquid carrier or a finely divided solid carrier, and/or both, and then, if needed, shaping the product into a desired formulation.

Typical compositions of the invention contain compound from about 90 to about 80% by weight, from about 80 to about 70% by weight, from about 70 to about 60% by weight, from about 60 to about 50% by weight, from about 50 to about 40% by weight, from about 40 to about 30% by weight, from about 30 to 20% by weight, from about 20 to about 10% by weight, from about 10 to about 4% by weight, from about 4.0% to about 2.0% by weight, from about 2.0% to about 1.0% by weight, and even from about 1.0% to about 0.0001% by weight.

It should be understood that the ingredients particularly mentioned above are merely examples and that some embodiments of formulations comprising the compositions of the present invention include other suitable components and agents. The invention further includes packages, vessels, or any other type of container that contain a compound of the present invention.

### General Synthesis Scheme and Examples

The invention can be further illustrated by the following synthetic schemes, although it will be understood that these examples are included merely for purposes of illustration and are not intended to limit the scope of the invention in any way unless otherwise specifically indicated.

Scheme 1 depicts a compound set claimed by this invention for use as a diagnostic agent. The synthesis begins with the di-halogenation of L-tyrosine, followed by di-protection of the amino acid functionality. Next, these di-protected amino acid analogs get coupled to form an ether linkage. Lastly, the linked di-protected compounds are de-protected to afford a wide range of novel deprotected compounds (in this example, the compound is an HCl salt) where R, R₁, R₂, R₃, R₄═H; where R₅ and R₆ are halogen; where R₇=—O—(CH₂)_{y}-Group.

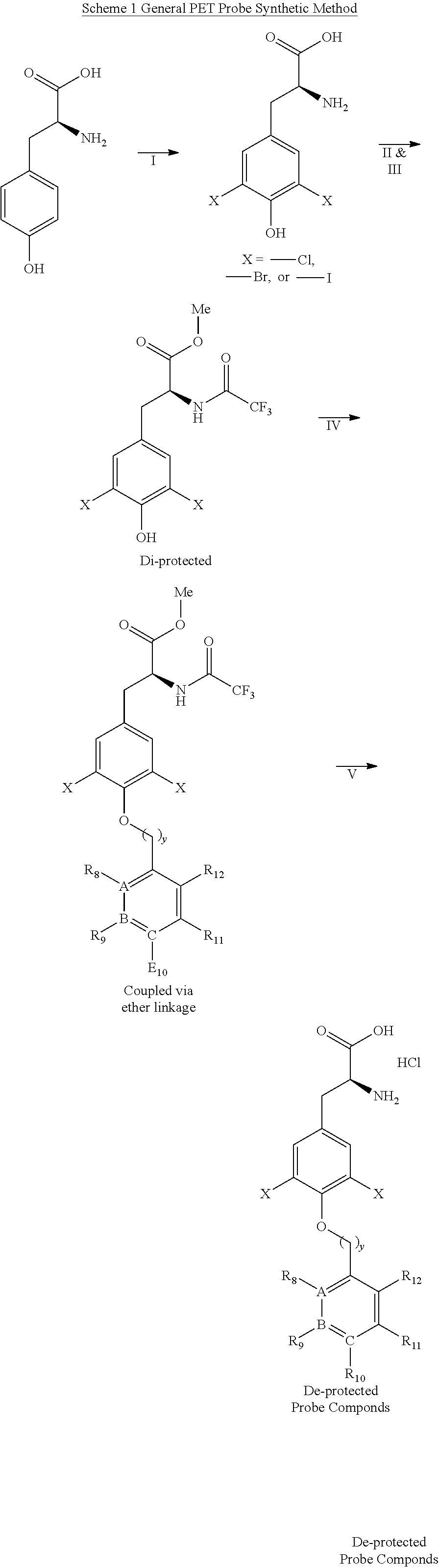

### Example

Various compounds may be prepared according to Scheme 1. For example, step-four: Di-iodo-tyrosine-N-TFA-O-Me (543.02 g/mol; 1000 mg; 1.84 mmol), (4-chloro-2-methoxybenzyl alcohol (334 mg; 1.93 mmol), and triphenylphosphine (262.29 g/mol; 960 mg; 3.68 mmol) were weighed out into a dry 100 mL round bottom flask containing a stir-bar. Next, anhydrous THF (25 mL) was added and capped with a sure-seal. The contents were stirred into solution and then cooled in an ice-bath (15-20 min). Then diisopropyl azodicarboxylate (800 μL; FW 202.21 g/mol; D=1.043 g/mL; 0.834 mg; 4.12 mmol) was added drop-wise and the contents stirred and warmed to room temperature (RT). The reaction was monitored by TLC. Upon completion, the reaction mixture was concentrated under reduced pressure and purified via SiO₂ column chromatography to afford light yellow solid (88% yield). Step-five: (S)-methyl 3-(4-(4-chloro-2-methoxybenzyloxy)-3,5-diiodophenyl)-2-(2,2,2-trifluoroacetamido)propanoate was dissolved in THF (5-6 mL) and stirred into solution. The solution was cooled in an ice bath (15-20 min) and then 15 mL of cold 1.0 M LiOH was added drop-wise. The contents were allowed to warm to RT and stirred over-night. The contents were again cooled into an ice bath (15-20 min). Next, the solution pH was adjusted to pH ˜3.0 with 3.0M HCl. The precipitant formed was collected via Büchner Filtration and subsequently dried under vacuum (54% yield; PET-42).

The product(s) of the process may be isolated using methods known to those of skill in the art, e.g., extraction, filtration, or crystallization. If necessary, compounds may be further purified using methods known to those of skill in the art, e.g., extraction, chromatography, distillation, or crystallization.

### Analysis of LAT1 and LAT2

The establishment and characterization of mammalian cell lines expressing human L-type amino acid transporterss (i.e. S2-hLAT1 and S2-hLAT2) has been previously described (Morimoto et al., Journal of Pharmacological Sciences, 2008, 108, 505-516).

General test procedures are as follows: All test compounds were dissolved in dimethyl sulfoxide (DMSO) to prepare stock solutions at the concentrations of 0.05, 0.5, 5.0 and 50 mM; next, these stock solutions were used and diluted with uptake solution to make final test concentrations of 0.10, 1.00, 10.0 and 100 μM, respectively. Therefore, all compounds used for uptake experiment were in uptake solution containing 0.2% DMSO; experiments are performed in triplicate for each concentration. For control uptake, only DMSO was added into the uptake solution to a final concentration of 0.2%.

S2-LAT1 and S2-LAT2 cells were seeded (1.3×10⁵ cells/well) into 24-well plates and cultured at 33° C. (5% CO₂) for 2-3 days until confluent (90%-100% confluence). Uptake experiments were performed at 37° C. The culture medium was removed and washed 3 times with uptake solution (37° C.; Hank's Na⁺ free buffer consisting of 125 mM choline chloride, 4.8 mM KCl, 1.3 mM CaCl₂, 1.2 mM MgCl₂, 25.0 mM HEPES, and 5.0 mM Tris, pH 7.4, supplemented with D-glucose (5.6 mM). The cells were equilibrated in uptake solution (300 μL; 37° C.) for 12 min. The equilibrated solution was removed and uptake solution containing radio-labeled compound (1.0 μM L-[¹⁴C] Leucine) with or without test compound (300 μL) was added to the cells (1.0 min). The test solution was removed and the cells washed 3 times with ice-cold uptake solution. The cells are then solubilized with 0.1N NaOH (0.5 mL) and transferred to scintillation vials. Liquid scintillation fluid (3.0 mL; aquasol-2) was added, mixed and substrate accumulation was measured by counting radioactivity via liquid scintillation counting (LSC; LSC6100, Aloka, Tokyo). The uptake value and % of inhibition were calculated to afford IC₅₀ values.

The IC₅₀ data for various compounds has been summarized in Table 1; it will be understood that these are merely examples for purposes of invention illustration and are not intended to limit the scope of the invention in any way unless otherwise specifically indicated.

TABLE 1
List of compounds and their S2-LAT1 and S2-LAT2 IC₅₀ μM data

| Comp. | | | | | | | | | | | | IC₅₀ (μM) | |
|---|---|---|---|---|---|---|---|---|---|---|---|---|---|
| # | X | n | Y | R8 | R9 | R10 | R11 | R12 | A | B | C | S2-LAT1 | S2-LAT2 |
| 1 | I | 3 | H | LP | H | H | H | H | N | C | C | NI | NI |
| 2 | I | 2 | H | LP | H | H | H | H | N | C | C | 27.3 | NI |
| 3 | I | 1 | H | LP | H | H | H | H | N | C | C | 8.0 | NI |
| 4 | I | 1 | H | H | LP | H | H | H | C | N | C | 86.0 | NI |
| 5 | I | 1 | H | H | H | LP | H | H | C | C | N | 57.0 | NI |
| 6 | I | 2 | H | H | H | O—Me | H | H | C | C | C | 4.4 | NI |
| 7 | I | 2 | H | H | O—Me | H | H | H | C | C | C | 7.1 | NI |
| 8 | I | 2 | H | O—Me | H | H | H | H | C | C | C | 3.9 | NI |
| 9 | I | 1 | H | O—Me | H | H | H | H | C | C | C | 17.8 | NI |
| 10 | I | 1 | H | H | O—Me | H | H | H | C | C | C | NI | NI |
| 11 | I | 1 | H | H | H | O—Me | H | H | C | C | C | 18.6 | NI |
| 12 | I | 2 | H | H | O—Me | O—Me | H | H | C | C | C | 4.8 | NI |
| 13 | I | 1 | H | H | O—Me | O—Me | H | H | C | C | C | 19.2 | 95.6 |
| 14 | I | 2 | H | H | H | CF₃ | H | H | C | C | C | 7.4 | NI |
| 15 | I | 2 | H | H | CF₃ | H | H | H | C | C | C | NI | NI |
| 16 | I | 2 | H | CF₃ | H | H | H | H | C | C | C | NI | NI |
| 17 | I | 1 | H | CF₃ | H | H | H | H | C | C | C | 122 | NI |
| 18 | I | 1 | H | H | CF₃ | H | H | H | C | C | C | 40.5 | NI |
| 19 | I | 1 | H | H | H | CF₃ | H | H | C | C | C | 84.7 | 7.7 |
| 20 | I | 1 | H | CH₃ | CH₃ | CH₃ | CH₃ | CH₃ | C | C | C | NI | NI |
| 21 | I | 1 | H | CF₃ | H | H | CF₃ | H | C | C | C | NI | NI |
| 22 | I | 1 | H | H | CF₃ | H | CF₃ | H | C | C | C | NI | NI |
| 23 | I | 2 | H | H | H | F | H | H | C | C | C | NI | NI |
| 24 | I | 2 | H | H | F | H | H | H | C | C | C | 67.0 | NI |
| 25 | I | 2 | H | F | H | H | H | H | C | C | C | 54.5 | NI |
| 26 | I | 1 | H | F | H | H | H | H | C | C | C | 29.8 | 7.4 |
| 27 | I | 1 | H | H | F | H | H | H | C | C | C | 42.5 | 12.9 |
| 28 | I | 1 | H | H | H | F | H | H | C | C | C | 10.0 | 11.1 |
| 29 | I | 1 | H | F | F | H | H | H | C | C | C | 27.0 | 13.9 |
| 30 | I | 1 | H | F | H | F | H | H | C | C | C | 14.0 | 5.5 |
| 31 | I | 1 | H | H | F | F | F | H | C | C | C | 17.3 | 30.6 |
| 31 | I | 1 | H | F | F | F | F | F | C | C | C | 48.3 | NI |
| 33 | I | 2 | H | H | H | O—CF₃ | H | H | C | C | C | NI | NI |
| 34 | I | 2 (CH2CO) | H | H | H | H | H | H | C | C | C | 11.9 | 47.9 |
| 35 | I | 2 (CH2CH(Ome)) | H | H | H | H | H | H | C | C | C | 8.8 | NI |
| 36 | I | 2 | H | H | H | NO₂ | H | H | C | C | C | 28.2 | NI |
| 37 | I | 1 | H | H | H | NO₂ | H | H | C | C | C | 11.5 | 8.2 |
| 38 | I | 1 | H | H | NO₂ | O—Me | H | H | C | C | C | 64.5 | NI |
| 39 | I | 1 | H | O—Me | O—Me | H | H | H | C | C | C | 52.8 | NI |
| 40 | I | 1 | H | O—Me | LP | O—Me | H | H | C | N | C | 15.4 | NI |
| 41 | I | 1 | H | O—Me | H | O—Me | H | H | C | C | C | 5.0 | NI |
| 42 | I | 1 | H | O—Me | H | Cl | H | H | C | C | C | 17.7 | NI |
| 43 | I | 1 | H | O—Me | O—Me | O—Me | H | H | C | C | C | 23.6 | NI |
| 44 | I | 1 | H | Me | Me | O—Me | H | H | C | C | C | 7.7 | NI |
| 45 | I | 1 | H | H | O—Me | O—Me | O—Me | H | C | C | C | 15.0 | NI |
| 46 | I | 1 | H | O—Me | H | O—Me | O—Me | H | C | C | C | 70.6 | NI |
| 47 | I | 1 | H | H | O—Et | O—Et | O—Et | H | C | C | C | 6.2 | NI |
| 48 | I | 1 | H | O—Et | H | O—Et | H | H | C | C | C | 17.2 | NI |
| 49 | I | 1 | H | H | O—Et | O—Et | H | H | C | C | C | 5.5 | NI |
| 50 | I | 1 | H | H | O—CH2— | O | H | H | C | C | C | 24.1 | NI |
| 51 | I | 1 | H | H | H | Cl | H | H | C | C | C | 10.4 | 29.5 |
| 52 | Br | 2 | H | H | H | O—Me | H | H | C | C | C | 6.8 | NI |
| 53 | Br | 2 | H | H | O—Me | H | H | H | C | C | C | 6.3 | NI |
| 54 | Br | 2 | H | O—Me | H | H | H | H | C | C | C | 5.4 | NI |
| 55 | Br | 1 | H | O—Me | H | H | H | H | C | C | C | 10.2 | NI |
| 56 | Br | 1 | H | H | O—Me | H | H | H | C | C | C | 67.1 | NI |
| 57 | Br | 1 | H | H | H | O—Me | H | H | C | C | C | 16.4 | 86.5 |
| 58 | Br | 2 | H | H | H | CF₃ | H | H | C | C | C | 43.2 | NI |
| 59 | Br | 2 | H | H | CF₃ | H | H | H | C | C | C | 31.8 | NI |
| 60 | Br | 2 | H | CF₃ | H | H | H | H | C | C | C | NI | NI |
| 61 | Br | 1 | H | CF₃ | H | H | H | H | C | C | C | NI | NI |
| 62 | Br | 1 | H | H | CF₃ | H | H | H | C | C | C | NI | NI |
| 63 | Br | 1 | H | H | H | CF₃ | H | H | C | C | C | NI | NI |
| 64 | Br | 2 | H | H | H | NO₂ | H | H | C | C | C | 19.5 | NI |
| 65 | Br | 1 | H | H | H | NO₂ | H | H | C | C | C | 29.2 | 5.5 |
| 66 | Br | 2 | H | H | H | F | H | H | C | C | C | 42.3 | NI |
| 67 | Br | 2 | H | H | F | H | H | H | C | C | C | 22.2 | NI |
| 68 | Br | 2 | H | F | H | H | H | H | C | C | C | 37.1 | 91.9 |
| 69 | Br | 1 | H | F | H | H | H | H | C | C | C | NI | 69.1 |
| 70 | Br | 1 | H | H | F | H | H | H | C | C | C | 71.5 | 49.3 |
| 71 | Br | 1 | H | H | H | F | H | H | C | C | C | 33.4 | 42.5 |
| 72 | Br | 2 | H | H | H | O—CF₃ | H | H | C | C | C | 8.3 | NI |
| 73 | Br | 2 (CH2CO) | H | H | H | H | H | H | C | C | C | 13.0 | 89.8 |
| 74 | Br | 2 (CH2CH(Ome)) | H | H | H | H | H | H | C | C | C | 38.5 | NI |
| 75 | Br | 2 | H | H | H | H | H | H | C | C | C | 19.9 | NI |
| 76 | Br | 2 | H | H | O—Me | O—Me | H | H | C | C | C | 3.9 | NI |
| 77 | Br | 1 | H | H | O—Me | O—Me | H | H | C | C | C | 23.3 | 130 |
| 78 | Br | 1 | H | O—Me | H | O—Me | H | H | C | C | C | 0.42 | 3.3 |
| 79 | Br | 1 | H | O—Me | O—Me | H | H | H | C | C | C | 27.5 | NI |
| 80 | Br | 1 | H | O—Me | H | O—Me | O—Me | H | C | C | C | 31.1 | NI |
| 81 | Br | 1 | H | O—Me | O—Me | O—Me | H | H | C | C | C | 12.1 | NI |
| 82 | Br | 1 | H | O—Me | LP | O—Me | H | H | C | N | C | 5.2 | 87.5 |
| 83 | Br | 1 | H | Me | Me | O—Me | H | H | C | C | C | 18.1 | NI |
| 84 | Br | 1 | H | H | O—Me | O—Me | O—Me | H | C | C | C | 9.4 | NI |
| 85 | Br | 1 | H | H | O—Et | O—Et | O—Et | H | C | C | C | 2.1 | NI |
| 86 | Br | 1 | H | H | O—Et | O—Et | H | H | C | C | C | 2.6 | 35.7 |
| 87 | Br | 1 | H | O—Et | H | O—Et | H | H | C | C | C | 1.4 | 28.7 |
| 88 | Br | 1 | H | H | O—CH2— | O | H | H | C | C | C | 22.0 | NI |
| 89 | Br | 1 | H | H | NO₂ | O—Me | H | H | C | C | C | NI | NI |
| 90 | Br | 1 | H | LP | H | H | H | H | N | C | C | NI | NI |
| 91 | Br | 1 | H | H | LP | H | H | H | C | N | C | NI | NI |
| 92 | Br | 1 | H | H | H | LP | H | H | C | C | N | NI | NI |
| 93 | Br | 1 | H | H | H | Cl | H | H | C | C | C | 15.5 | 40.3 |
| 94 | Br | 1 | H | CF₃ | H | H | CF₃ | H | C | C | C | NI | NI |
| 95 | Br | 1 | H | F | F | H | H | H | C | C | C | 88.0 | 42.2 |
| 96 | Br | 1 | H | H | F | F | F | H | C | C | C | 59.5 | NI |
| 97 | Br | 1 | H | F | H | F | H | H | C | C | C | 38.5 | 43.8 |
| 98 | Br | 1 | H | O—Me | H | Cl | H | H | C | C | C | 6.7 | NI |
| 99 | Cl | 2 | H | H | H | CF₃ | H | H | C | C | C | 1.3 | NI |
| 100 | Cl | 2 | H | H | CF₃ | H | H | H | C | C | C | 13.2 | NI |
| 101 | Cl | 2 | H | CF₃ | H | H | H | H | C | C | C | NI | NI |
| 102 | Cl | 1 | H | CF₃ | H | H | H | H | C | C | C | 19.4 | NI |
| 103 | Cl | 1 | H | H | CF₃ | H | H | H | C | C | C | 2.9 | 9.2 |
| 104 | Cl | 1 | H | H | H | CF₃ | H | H | C | C | C | 10.1 | 14.4 |
| 105 | Cl | 2 | H | H | H | F | H | H | C | C | C | 34.5 | NI |
| 106 | Cl | 2 | H | H | F | H | H | H | C | C | C | 37.4 | 61.3 |
| 107 | Cl | 2 | H | F | H | H | H | H | C | C | C | 35.1 | 56.0 |
| 108 | Cl | 1 | H | H | F | F | H | H | C | C | C | NI | NI |
| 109 | Cl | 1 | H | H | H | F | H | H | C | C | C | 10.2 | 19.0 |
| 110 | Cl | 1 | H | LP | H | H | H | H | N | C | C | 93.3 | NI |
| 111 | Cl | 1 | H | H | LP | H | H | H | C | N | C | 106 | NI |
| 112 | Cl | 1 | H | H | H | LP | H | H | C | C | N | NI | NI |
| 113 | Cl | 2 | H | H | H | O—Me | H | H | C | C | C | 25.3 | NI |
| 114 | Cl | 2 | H | H | O—Me | H | H | H | C | C | C | 4.2 | 86.7 |
| 115 | Cl | 2 | H | O—Me | H | H | H | H | C | C | C | 3.4 | NI |
| 116 | Cl | 1 | H | O—Me | H | H | H | H | C | C | C | 5.8 | 93.6 |
| 117 | Cl | 1 | H | H | O—Me | H | H | H | C | C | C | 13.6 | 28.3 |
| 118 | Cl | 1 | H | H | H | O—Me | H | H | C | C | C | 16.8 | 59.7 |
| 119 | Cl | 1 | H | H | H | NO₂ | H | H | C | C | C | 62.4 | 23.1 |
| 120 | Cl | 2 | H | H | H | NO₂ | H | H | C | C | C | 19.8 | NI |
| 121 | Cl | 2 | H | H | H | O—CF₃ | H | H | C | C | C | 7.7 | NI |
| 122 | Cl | 2 | H | H | H | H | H | H | C | C | C | 50.7 | NI |
| 123 | Cl | 2 (CH2CO) | H | H | H | H | H | H | C | C | C | ND | ND |
| 124 | Cl | 2 (CH2CH(Ome)) | H | H | H | H | H | H | C | C | C | NI | NI |
| 125 | Cl | 2 | H | H | H | H | H | H | C | C | C | 50.7 | NI |
| 126 | Cl | 2 (CH2CO) | H | H | H | H | H | H | C | C | C | ND | ND |
| 127 | Cl | 2 (CH2CH(Ome)) | H | H | H | H | H | H | C | C | C | NI | NI |
| 128 | Cl | 2 | H | H | O—Me | O—Me | H | H | C | C | C | ND | ND |
| 129 | Cl | 1 | H | H | O—Me | O—Me | H | H | C | C | C | 17.8 | NI |
| 130 | Cl | 1 | H | H | O—CH2— | O | H | H | C | C | C | 4.2 | 27.3 |
| 131 | Cl | 1 | H | H | NO2 | O—Me | H | H | C | C | C | 8.4 | 126 |
| 132 | Cl | 1 | H | O—Me | O—Me | H | H | H | C | C | C | 28.2 | NI |
| 133 | Cl | 1 | H | O—Me | H | O—Me | H | H | C | C | C | 0.8 | 5.5 |
| 134 | Cl | 1 | H | O—Me | H | Cl | H | H | C | C | C | 4.4 | NI |
| 135 | Cl | 1 | H | O—Me | LP | O—Me | H | H | C | N | C | 2.4 | 32.5 |
| 136 | Cl | 1 | H | O—Me | O—Me | O—Me | H | H | C | C | C | 8.1 | NI |
| 137 | Cl | 1 | H | Me | Me | O—Me | H | H | C | C | C | 3.5 | NI |
| 138 | Cl | 1 | H | O—Me | H | O—Me | O—Me | H | C | C | C | 29.2 | NI |
| 139 | Cl | 1 | H | H | O—Et | O—Et | O—Et | H | C | C | C | 1.2 | NI |
| 140 | Cl | 1 | H | O—Et | H | O—Et | H | H | C | C | C | 2.2 | NI |
| 141 | Cl | 1 | H | H | O—Et | O—Et | H | H | C | C | C | 2.9 | 44.9 |
| 142 | M—Cl | 1 | H | H | O—Me | O—Me | H | H | C | C | C | NI | NI |
| 143 | M—Cl | 2 | H | H | H | O—Me | H | H | C | C | C | NI | NI |
| 144 | Cl | 1 | * Me | H | O—CH2— | O | H | H | C | C | C | NI | NI |
| 145 | Cl | 1 | * Me | H | H | CF3 | H | H | C | C | C | 100 | NI |
| 146 | Cl | 1 | * Me | H | H | O—Me | H | H | C | C | C | 6.7 | 16.4 |
| 147 | Cl | 1 | * Me | H | O—Me | H | H | H | C | C | C | NI | NI |
| 148 | Cl | 1 | * Me | O—Me | H | H | H | H | C | C | C | NI | NI |
| 149 | Cl | 1 | * Me | O—Me | O—Me | H | H | H | C | C | C | NI | NI |
| 150 | Cl | 1 | * Me | H | O—Me | O—Me | H | H | C | C | C | NI | NI |
| 151 | Cl | 1 | * Me | H | O—Me | O—Me | O—Me | H | C | C | C | NI | NI |
| 152 | Cl | 1 | * Me | H | H | LP | H | H | C | C | N | NI | NI |

NI = no inhibition at 100 μM;
* racemic mixture;
LP = lone pair of electrons

The data summarized in Table 1 are informative and illustrate that the structure-activity relationship differences between LAT1 and LAT2 are subtle yet complex. The data illustrate: i) amino acid functionality is required for binding; ii) stereochemistry is important; and iii) that an important hydrogen bonding region exists about 10 angstroms from the alpha amino acid carbon. In addition to a preference for aromatic amino acids, size and electronic effects on the aromatic ring influence binding and selectivity. These data also provide examples of preferred compounds; for example, 3, 6-8, 12, 14, 35, 41, 44, 47, 49, 72, 76, 85, 98, 99, 115, 121, 134, 137, 139, 140 and the like. Furthermore, it will be understood that these are merely examples for purposes of invention illustration and are not intended to limit the scope of the invention in any way.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A compound represented by the general formula I: 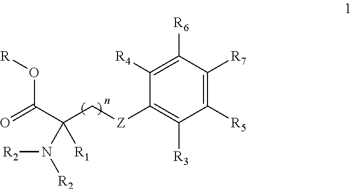
a pharmaceutically acceptable salt or ester thereof, a solvate thereof, a chelate thereof, a non-covalent complex thereof, a pro-drug thereof, a radio-labeled analog thereof (i.e. ¹⁸F, ¹²⁴I, ¹¹C, etc), and mixtures of any of the foregoing, wherein:
R is selected from hydrogen, C₁-C₂₂ alkyl, C₄-C₂₂ alkenyl, C₄-C₂₀ dienyl, C₆-C₂₂ trienyl, C₈-C₂₂ tetraenyl, a polyethylene glycol, a polypropylene glycol, or co-blocked polymer;
R¹ is selected from hydrogen, deuterium, tritium, methyl (—CH₃), —CH₂F, —CHF₂, or —CF₃;
R² is individually selected from hydrogen, methyl, or ethyl;
n is 0, 1, 2, or 3 methylene (—CH₂—) units;
Z is selected from O, N, NR′, S, SO, SO₂, —SO₂NR′—, —NR′SO₂—, wherein R′ is selected from hydrogen, alkyl, substituted alkyl, cycloalkyl, substituted cycloalkyl, aryl, substituted aryl, aralkyl and substituted aralkyl; or even where Z is absent (when n=0 and Z absent, then directly connected to the aromatic ring).
R³ and R⁴ are individually selected from hydrogen, deuterium, tritium, —Cl, —Br, —I, —F, —OH, —OR′, C1-C4 alkyl or substituted alkyl, —NH₂, —NHR′, —N(R′)₂, —NHSO₂R′, —NR₈SO₂R′, —SO₂NH₂, —SO₂NHR′, —SO₂N(R′)₂, wherein R′ has been previously defined;
R⁵, R⁶, and R⁷ are individually selected from hydrogen, deuterium, tritium, —Cl, —Br, —I, —F, —OH, —OR′, alkyl and substituted alkyl (C1-C4), —NH₂, —NHR′, —N(R′)₂, —NHSO₂R′, —NR′SO₂R′, —SO₂NH₂, —SO₂NHR′, —SO₂N(R′)₂, wherein R′ has been previously defined, or from the group consisting of C₃-C₂₄ alkyl, C₃-C₂₄ alkenyl, C₄-C₂₄ dienyl, C₆-C₂₄ trienyl, C₈-C₂₄ tetraenyl and mixtures thereof, C₆-C₁₈ aryl, substituted C₆-C₁₈ aryl, C₁-C₁₄-alkoxy, halogen, carboxy, cyano, C₁-C₁₄-alkanoyloxy, C₁-C₁₄-alkylthio, C₁-C₁₄-alkylsulfonyl, C₂-C₁₄-alkoxycarbonyl, C₂-C₁₄-alkanoylamino, —O—R⁸, S—R⁸, —SO₂—R⁸, —NHSO₂R⁸ and —NHCO₂R⁸, wherein R⁸ is phenyl, naphthyl, or phenyl or naphthly substituted with one to three groups selected from C₁-C₆-alkyl, C₆-C₁₀ aryl, C₁-C₆-alkoxy and halogen, and C₄-C₂₀ hydroxyheteroaryl wherein the heteroatoms are selected from the group consisting of sulfur, nitrogen, and oxygen;

2. A composition according to claim 1, wherein the compound is present in an amount of at least 0.0001% by weight.

3. The composition according to claim 2, wherein the compound is present in an amount of from about 0.0001% to about 0.1% by weight.

4. The composition according to claim 2, wherein the compound is present in an amount of from about 0.1% to about 5% by weight.

5. The composition according to claim 2, wherein the compound is present in an amount of from about 5% to about 50% by weight.

6. The composition according to claim 2, wherein the compound is present in an amount of from about 50% to about 90% by weight.

7. A method to diagnose or monitor a condition comprising the administration of a compound according to claim 1.

8. A method of claim 7, wherein the subject is a mammal.

9. A method of claim 8, where the subject is a human.

10. A kit comprising:
A composition comprising at least one compound of claim 1,
Instructions for administrating the composition comprising at least one compound of claim 1 to a human or mammal.

11. A container, wherein the contents of the container comprise:
At least one compound of claim 1,
Wherein the container contains, is labeled, or is otherwise accompanied by instructions for administration to a human or mammal in a manner that results in interacting with selected cells, tissues, or organs for a selected period of time.
